(19) United States
(12) Patent Application Publication
(10) Pub. No.: US 2012/0015426 A1
(43) Pub. Date: Jan. 19, 2012

(54) BACTERIA FOR HIGH EFFICIENCY CLONING

(75) Inventors: Fredric R. BLOOM, Germantown, MD (US); Brian Schmidt, Rockville, MD (US)

(73) Assignee: LIFE TECHNOLOGIES CORPORATION, Carlsbad, CA (US)

(21) Appl. No.: 13/246,623

(22) Filed: Sep. 27, 2011

Related U.S. Application Data
(63) Continuation of application No. 12/646,828, filed on Dec. 23, 2009.
Application No. 12/646,828 is a continuation of application No. 10/542,628, filed on May 22, 2006, now abandoned.
(60) Provisional application No. 60/440,333, filed on Jan. 16, 2003.

(51) Int. Cl. C12N 1/20

(52) U.S. Cl. 435/252.8; 435/252.1

(57) ABSTRACT

Disclosed are novel bacterial hosts that are capable of high efficiency transformation with methylated and/or unmethylated nucleic acids, and that are bacteriophage resistant. Such bacteria contain: (1) an F′ episome that confers high efficiency transformability; (2) one or more mutations that allow transformation of methylated nucleic acids; (3) one or more mutations that allow transformation with unmethylated nucleic acids; and/or (4) one or more mutations that confer resistance to bacteriophage infection. Also disclosed are methods for transforming such bacteria, and kits that contain such bacteria (e.g., that have been made competent for transformation).

### BACKGROUND OF THE INVENTION

1. Field of the Invention

This invention relates to the biotechnology field. In particular, the invention relates to bacteriophage resistant bacteria that are capable of high efficiency transformation with methylated and/or unmethylated nucleic acids.

2. Related Art

Cloning operations in the biotechnology field often involve introducing exogenous nucleic acids into a bacterial host. Specially designed bacterial hosts can help biotechnology researchers meet the challenges associated with such cloning operations.

One particular cloning challenge relates to transforming bacterial hosts with nucleic acids that are present in low abundance. Transformation efficiency can be affected by the genotype of the bacterial host. Thus, using a bacterial host that is capable of high efficiency transformation can increase the odds of cloning small amounts of nucleic acids. Such high efficiency hosts are particularly useful for obtaining rare nucleic acids as clones in plasmid libraries (e.g., cDNA libraries). Such hosts also are particularly useful for transforming the products of inefficient or complex cloning procedures (e.g., single or multiple blunt ended ligation reactions).

Another cloning challenge relates to bacterial restriction systems that preclude the introduction and maintenance of methylated nucleic acids in bacterial hosts. In general, methylated DNA is associated with rarely expressed genomic DNA (rarely expressed genomic DNA tends to be methylated to a greater degree than actively expressed genomic DNA, which may or may not be methylated). Because methylated DNA often is expressed only during particular stages of development or in particular cell types (e.g., in particular tissues or in diseased cells), it can be of particular interest to biotechnology researchers. Bacterial hosts that do not prevent the introduction and maintenance of methylated DNA are useful for cloning such interesting DNA.

Another cloning challenge relates to bacteriophage infection of transformed bacteria. Bacteriophage transmitted by aerosolization (e.g., bacteriophage T1) are a particularly serious threat to transformed bacteria, including the numerous strains created and maintained in high throughput biotechnology laboratories and genomics research facilities. Bacterial hosts that are resistant to bacteriophage infection are useful to prevent infection and destruction of such valuable transformed bacteria.

Many commercially available bacterial hosts are unable to clone methylated DNA and are susceptible to bacteriophage infection.

### SUMMARY OF THE INVENTION

The invention features bacterial hosts that are capable of high efficiency transformation with methylated and/or unmethylated nucleic acids, and that are resistant to bacteriophage infection. In particular, the invention features bacteria that contain: (1) an F′ episome that confers high efficiency transformability; (2) one or more mutations that allow transformation of methylated nucleic acids; (3) one or mutations that allow transformation with unmethylated nucleic acids; and/or (4) one or more mutations that confer resistance to bacteriophage infection. In a preferred aspect, the bacterial host is a Escherichia coli K-12 bacterium having a genotype comprising mcrA Δ(mrr-hsdRMS-mcrBC) tonA/F′ proAB⁺ lacI^{q} lacZΔM15 Tn10(Tet^{R}). In a particularly preferred aspect, the bacteria is E. coli K-12 strain BRL3946, having genotype: mcrA Δ(mrr-hsdRMS-mcrBC) φ80(lacZ)ΔM15 Δ(lacZYA-argF) U169 endA1 recA1 supE44 thi-1 gyrA96 relA1 deoR tonA panD/F′ proAB⁺ lacI^{q} lacZΔM15 Tn10(Tet^{R}). The BRL3946 strain is deposited in the Agricultural Research Service Patent Culture Collection maintained by the National Center for Agricultural Utilization Research in Peoria, Ill., USA (NRRL accession No. B-30640). The invention also features, methods for transforming such bacteria. The invention also features kits that contain such bacteria (e.g., having been made competent for transformation).

Bacterial hosts in accord with the invention satisfy several needs in the art. Such bacteria are capable of high efficiency transformation, enabling the cloning of nucleic acids that are present in low abundance. Such bacteria also enable the cloning of methylated DNA, including rarely expressed genomic DNA. In addition, such bacteria are resistant to bacteriophage infection, protecting transformants from infection and destruction.

Other useful properties and advantages of the invention will be apparent from the following detailed description, and from the claims. The disclosed materials, methods, and examples are illustrative only and are not intended to be limiting. Skilled artisans will appreciate that methods and materials similar or equivalent to those described herein can be used to practice the invention.

Unless otherwise defined, all technical and scientific terms used herein have the meaning commonly understood by one skilled in the biotechnology art. All publications, patent applications, patents, and other references mentioned herein are incorporated by reference in their entirety. In case of conflict, the present specification, including definitions, will control

### DETAILED DESCRIPTION OF THE INVENTION

The invention provides methods and materials for cloning nucleic acids. In particular, the invention provides novel bacterial hosts that are capable of high efficiency transformation with methylated and/or unmethylated nucleic acids, and that are bacteriophage resistant. The invention also provides methods for transforming such bacteria. The invention also provides kits that contain such bacteria (e.g., having been made competent for transformation).

### Bacterial Hosts

A bacterial host (or, interchangeably, “bacterium” or “host”) in accord with the invention contains: (1) an F′ episome that confers high efficiency transformability; (2) one or more mutations that allow transformation of methylated nucleic acids; (3) one or mutations that allow transformation with unmethylated nucleic acids; and/or (4) one or more mutations that confer resistance to bacteriophage infection.

Suitable bacterial hosts include gram negative and gram positive bacteria of any genus known to those skilled in the art, including Escherichia sp. (e.g., E. coli), Klebsiella sp., Streptomyces sp., Streptocococcus sp., Shigella sp., Staphylococcus sp., Erwinia sp., Klebsiella sp., Bacillus sp. (e.g., B. cereus, B. subtilis and B. megaterium), Serratia sp., Pseudomonas sp. (e.g., P. aeruginosa and P. syringae) and Salmonella sp. (e.g., S. typhi and S. typhimurium). Many bacterial strains and serotypes are suitable for the invention, including E. coli strains K, B, C, and W. A preferred bacterial host is E. coli strain K-12. Bacterial hosts in accord with the invention are isolated (i.e., separated at least partially from other bacteria and materials with which they are associated in nature).

Bacterial hosts in accord with the invention include those disclosed herein, as well as derivatives thereof. A “derivative” bacterium is described with reference to a specified “parent” or “ancestor” bacterium. A derivative bacterium can be made by introducing one or more mutations (e.g., addition, insertion, deletion or substitution of one or more nucleic acids) in the chromosome of a specified bacterium. For example, one or more of the E. coli K-12 nucleic acid open reading frames identified in RefSeq: NC_{—}000913 (derived from GenBank: U00096, both herein incorporated by reference) can be subjected to mutagenesis. A derivative bacterium also can be made by introducing one or more mutations (e.g., addition, insertion, deletion or substitution of one or more nucleic acids) in an extrachromosomal nucleic acid present in a specified bacterium. A derivative bacterium can be made by adding one or more extrachromosomal nucleic acids (e.g., plasmid or F′ episome) to a specified bacterium. A derivative bacterium also can be made by removing (e.g., by “curing”) extrachromosomal nucleic acids from a specified bacterium. Techniques for making all such derivatives can be practiced as a matter of routine by those of skill in the art.

F′ episomes conferring high efficiency transformability. A bacterial host in accord with the invention can contain an F′ episome that enhances its transformation efficiency (or “transformability”). Transformation refers to the introduction and maintenance (transient or stable) of exogenous nucleic acids in a bacterium. Exogenous nucleic acids are nucleic acids from any source, natural or otherwise, that are capable of being introduced into a bacterium. Exogenous nucleic acids include, e.g., plasmid DNA and phage DNA.

An F′ episome that confers high efficiency transformability can increase the efficiency of transformation of a bacterial host by a factor greater than one, relative to a bacterium that lacks the F′ but otherwise has the same genotype. In preferred embodiments, an F′ episome increases the transformation efficiency of a bacterial host 2-4 fold, or even more than 4 fold. In general, a bacterial host in accord with the invention can be transformed with a transformation efficiency of at least 1×10⁷ (e.g., 1×10⁸, 1×10⁹) transformants per microgram of DNA.

In some embodiments, all or part of an F′ episome can be integrated into a host's chromosome. In some embodiments, all or part of an F′ episome can be present on a self-replicating DNA molecule. In some embodiments, all or part of an F′ episome can be linked genetically to a selectable marker (e.g., a selectable marker providing resistance to an antibiotic, such as a gene providing resistance to tetracycline). Preferred F′ episomes are disclosed in U.S. Pat. No. 6,274,369. Other suitable F′ episomes can be identified and introduced into bacterial cells by those of skill in the art using routine methods.

Transformation with Methylated and/or Unmethylated DNA.

A bacterial host in accord with the invention can contain one or more mutations that allow transformation of methylated and/or unmethylated nucleic acids. Such mutations abolish or interfere with the function of bacterial Restriction-Modification Systems (RMS) that degrade incoming exogenous DNA. A gene encoding an RMS protein is mutant if a mutation (e.g., addition, insertion, deletion, and/or substitution of one or more nucleic acids) involves one or more nucleotides that encode the RMS component, and/or cis-acting determinants that affect the transcription of such nucleotides. Those of skill in the art will recognize that antisense nucleic acids (e.g., produced from the bacterial chromosome or from self-replicating nucleic acids such as plasmids) can be used to disrupt the function of a bacterial RMS. Such antisense molecules are within the meaning of “mutation” as used herein. Antisense nucleic acids are described, e.g., in U.S. Pat. Nos. 5,254,678; 5,496,698; 5,525,468; 5,616,459; 5,874,414; 6,015,794; 5,631,115; 5,631,359; 5,646,020; 5,837,855; 6,022,962; 6,307,041; 5,652,094; 6,096,715; 6,140,491; 5,672,511; 5,767,263; 5,879,938; 5,891,684; 6,204,027; 5,545,729; 5,942,395; 6,265,167; 5,908,779; 5,994,124; and 5,650,502. Other, siRNA-type, antisense nucleic acids are described in, e.g., U.S. Pat. Nos. 6,326,193, 5,795,715, and 5,457,026; and U.S. Patent Applications 2002/0007051 A1, 60/068,562 and Ser. No. 09/215,257 (priority documents for WO9932619), 60/159,776 and 60/193,218 (priority documents for WO0129058), 60/189,739 and 60/243,097 (priority documents for WO0168836), and 60/193,594 and 60/265,232 (priority documents for WO0175164).

In general, bacteria have two types of RMS. The first type of bacterial RMS degrades unmethylated DNA. This type of RMS is exemplified by the E. coli hsdR, hsdM and hsdS gene products, which form an enzyme complex that either cleaves or methylates a target site (in the hsd system, 5″-AAC[N₆]GTGC-3′). The enzyme cleaves unmethylated target sites, buts methylates target sequence that are hemimethylated. Thus, a bacterium that contains functional hsd gene products cleaves incoming exogenous DNA that is unmethylated at the target site. On the other hand, a bacterial host that contains one or more mutations in hsdR, hsdM and/or hsdS that result in an absent or non-functional hsd enzyme complex can be useful for cloning exogenous DNA that is unmethylated at the target site. In a preferred embodiment, a bacterial host in accord with the invention can be useful for cloning unmethylated DNA, by virtue of deletion of hsdR, hsdM and hsdS.

The second type of bacterial RMS degrades methylated DNA. This type of RMS is exemplified by two E. coli methylcytosine RMS, McrA and McrBC, and by the E. coli methyladenine RMS, Mrr. The McrA system degrades DNA methylated at the cytosine of the CG dinucleotide, and DNA methylated at the second cytosine of the sequence 5′-CCGG-3′. The McrBC system degrades DNA methylated at the cytosine of the sequence (^{G}/_{A})C. The Mrr system degrades adenine-methylated and/or cytosine-methylated DNA. Bacterial hosts that contain one or more mutations that result in an absent or non-functional McrA, McrBC, and/or Mrr RMS component can be useful for cloning methylated exogenous DNA. In a preferred embodiment, a bacterial host useful for cloning methylated DNA has mutant McrA, McrBC and Mrr genes.

RMS of both types in bacteria other than E. coli have been characterized and can be subject to mutation by those of skill in the art as a matter of routine to allow the introduction of methylated and/or unmethylated DNA, as desired.

Bacteriophage resistance. A bacterial host in accord with the invention can contain one or more mutations that confer resistance to bacteriophage infection. Bacteriophage (or “phage”) can be distinguished from each another based on their genetic composition and/or their virion morphology. Some phage have double stranded DNA genomes, including phage of the corticoviridae, lipothrixviridae, plasmaviridae, myrovridae, siphoviridae, sulfolobus shibate, podoviridae, tectiviridae and fuselloviridae families. Other phage have single stranded DNA genomes, including phage of the microviridae and inoviridae families. Other phage have RNA genomes, including phage of the leviviridae and cystoviridae families. Exemplary bacteriophage include phages Wphi, Mu, T1, T2, T3, T4, T5, T6, T7, P1, P2, P4, P22, fd, phi6, phi29, phiC31, phi80, phiX174, SP01, M13, MS2, PM2, SSV-1, L5, PRD1, Qbeta, lambda, UC-1, HK97 and HK022.

Host and phage proteins important for bacteriophage infection are known in the art and can be subject to mutation by those of skill in the art using routine methods. Bacteria resistant to phage infection also can be obtained by routine screening of mutant (spontaneous or induced) bacteria. Phage resistant bacteria often have cellular properties that inhibit or substantially reduce the ability of one or more types of bacteriophage to insert their genetic material into the bacterial cell. Thus, some bacteriophage resistant bacteria have cellular properties that prevent or inhibit bacteriophage attachment to the bacterial cell surface, and/or insertion of bacteriophage genetic material into the bacterial cytoplasm. Bacteriophage resistance is described further in, e.g., U.S. Pat. Nos. 5,538,864, 5,432,066 and 5,658,770, and in Saito, H. and Richardson, C. C., J. Virol. 37:343-352 (1981), Stacey, K. A. and Oliver, P., J. Gen. Microbiol. 98:569-578 (1977), Coulton, Biochim. Biophys. Acta 717:154-162 (1982), Carta, G. R. and Bryson, V., J. Bacteriol. 92:1055-1061 (1966), Ronen A. and Zehavi, A., J. Bacteriol. 99:784-790 (1969), Braun-Breton, C. and Hofnung, M., Mol. Gen. Genet. 16:143-149 (1978), and Picken, R. N. and Beacham, I. R., J. Gen. Microbiol. 102:305-318 (1977).

A protein important for phage infection is mutant if a mutation (e.g., addition, insertion, deletion, and/or substitution of one or more nucleic acids) involves one or more nucleotides that encode the protein, and/or cis-acting determinants that affect the transcription of such nucleotides. Those of skill in the art will recognize that antisense nucleic acids (e.g., produced from the bacterial chromosome or from self-replicating nucleic acids such as plasmids) can be used to disrupt bacteriophage infection. Such antisense molecules are within the meaning of “mutation” as used herein.

Bacteriophage transmitted by aerosolization are a particularly serious threat to transformed bacteria in biotechnology research laboratories, where some such phage (e.g., phage T1) can survive for years in contaminated ventilation ducts. Thus, in a preferred embodiment, a bacterial host of the invention is rendered resistant to phage T1 by mutation of the tonA gene, which encodes a non-essential iron transport protein that T1 exploits for attachment to the bacterial cell surface.

### Competence and Transformation Methods

Protocols for rendering bacteria capable of taking up and maintaining exogenous nucleic acids (i.e., making them “transformable” or “competent”), and for transforming them are well known and can be practiced as a matter or routine by those skilled in the art using bacterial hosts in accord with the invention. Protocols based on that disclosed in Hanahan, J. Mol. Biol. 166:557-580 (1983) typically result in transformation efficiencies of 10⁷ to 10⁹ transformants/μg of supercoiled plasmid DNA, depending on the bacterial host. Protocols based on that disclosed in Mandel and Higa, J. Mol. Bio. 53:159-162 (1970) typically yield 10⁵ to 10⁶ transformants/μg of supercoiled plasmid DNA. Bacterial hosts also can be transformed by electroporation.

Incubating a bacterial host in a solution that contains multivalent cations (e.g., calcium, manganese, magnesium and barium), sulfhydryl reagents and/or organic solvents can affect transformation efficiency. The temperature at which a bacterial host is grown prior to being rendered competent also can affect transformation efficiency. For example, E. coli hosts grown at temperatures between 25 and 30° C. can exhibit increased transformation efficiency relative to E. coli hosts grown at 37° C. (U.S. Pat. No. 4,981,797).

### Kits

The invention provides kits that include bacterial hosts disclosed herein. A bacterial host typically is provided in one or more sealed containers (e.g., packet, vial, tube, or microliter plate), which in some embodiments also can contain bacterial nutritional media. In some embodiments the bacterial host is provided in desiccated or lyophilized form. In some embodiments the bacterial host has been rendered competent for transformation. In some embodiments a kit includes sterile bacterial nutritional media in a separate container. A kit typically includes literature describing the properties of the bacterial host (e.g., its genotype) and/or instructions regarding its use for transformation. In some embodiments a kit includes one or more nucleic acids (e.g., plasmid and/or polymerase chain reaction primer) in a separate container. In some embodiments a kit includes one or more cloning enzymes (e.g., nucleic acid polymerase, nucleic acid ligase, nucleic acid topoisomerase, uracil DNA glycosylase, protease, phosphatase, ribonuclease, and/or ribonuclease inhibitor) in a separate container.

The invention is further described in the following examples, which serve to illustrate but not to limit the scope of the invention described in the claims.

### EXAMPLES

### Example 1

### BRL3496 Strain Construction

E. coli DH5α™ (Invitrogen) MCR cells {mcrA Δ(mrr-hsdRMS-mcrBC) φ80(lacZ)ΔM15 Δ(lacZYA-argF)U169 endA1 recA1 supE44 thi-1 gyrA96 relA1 deoR/F⁻} were transformed with plasmid pCM301 recA to yield DH5α™ MCR/pCM301 recA. Plasmid pCM301 recA has a temperature sensitive replicon and encodes a functional recA gene product, enabling P1 mediated transduction. Lysates from a P1CM lysogenized DH5α™ (Invitrogen) derivative having a 1.5 Kb insertion in the tonA (fhuA), a linked zad220:Tn10 transposon and a mutation in the panD gene were used to transduce strain DH5α™ (Invitrogen) MCR/pCM301 recA. Tetracycline resistant colonies were selected and screened for resistance to bacteriophage T5. Phage T5 resistant colonies were repurified on tetracycline-containing media at 42° C. to cure pCM301 recA. The resultant strain, designated BRL3932, has the genotype: mcrA Δ(mrr-hsdRMS-mcrBC) φ80(lacZ)ΔM15 Δ(lacZYA-argF) U169 endA1 recA1 supE44 thi-1 gyrA96 relA1 deoR tonA(T1) zad220::Tn10panD/F⁻.

Strain 3932 was cured of the Tn10 transposon by plating on fusaric acid-containing media and screening fusaric acid resistant colonies for tetracycline sensitivity. A tetracycline sensitive derivative of 3932 was selected. This strain, designated BRL3939, has the genotype: mcrA Δ(mrr-hsdRMS-mcrBC) φ80(lacZ)ΔM15 Δ(lacZYA-argF) U169 endA1 recA1 supE44 thi-1 gyrA96 relA1 deoR tonA(T1) panD/F. Several isolated colonies of BRL3939 were cross streaked against bacteriophage T5 and all were T5 resistant.

A DH10B™ (Invitrogen) derivative containing an F′ episome {mcrA Δ(mrr-hsdRMS-mcrBC) φ80lacZΔM115 ΔlacX74 deoR recA1 endA1 araΔ139 Δ(ara, leu)7697 galU galK rpsL (Str^{r}) nupG/F′ proAB⁺ lacI^{q} lacZΔM15 Tn10(Tet^{R})} was mated with BRL3939, and the mating mix was plated on Luira-Bertani media supplemented with tetracycline and naladixic acid. The resultant strain, designated BRL3946, has the genotype: mcrA Δ(mrr-hsdRMS-mcrBC) φ80(lacZ)ΔM15 Δ(lacZYA-argF) U169 endA1 recA1 supE44 thi-1 gyrA96 relA1 deoR tonA panD/F′ proAB⁺ lacI^{q} lacZ ΔM15 Tn10(Tet^{R}).

### Example 2

### Transforming BRL3496 with Plasmid DNA

pUC19 DNA (10 pg) was used to transform BL3496 and DH5α™ (Invitrogen) {F′ φ80(lacZ)ΔM15 Δ(lacZYA-argF) U169 endA1 recA1 hsdR17(rₖ⁻, mₖ⁺) phoA supE44 thi-1 gyrA96 recA1 deoR} cells rendered competent by the method described in U.S. Pat. No. 4,981,797. Transformation reactions were incubated 15 minutes on ice with DNA, heat shocked at 42° C. for 45 seconds, supplemented with 450 μl SOC medium, and incubated at 37° C. for 1 hour. The number of colony forming units determined to be present in each transformation reaction (i.e., per 10 pg pUC19 DNA) is as follows: 33440 CFU for BL3496 and 10500 CFU for DH5α™ (Invitrogen)

### Example 3

### Transforming BRL3496 with Methylated and Unmethylated DNA

pUC19 DNA (100 ng) was incubated with and without S-adenosyl-methionine (SAM) (1.6 mM) for an hour at 37° C. in the presence of four units of methylase in a 16 μl reaction volume. Reaction were diluted 100-fold and 2 μl was used to transform. BL3496 and DH5α™ (Invitrogen) cells rendered competent by the method described in U.S. Pat. No. 4,981,797. Transformation reactions were incubated 15 minutes on ice with DNA, heat shocked at 42° C. for 45 seconds, supplemented with 250 μl SOC medium, and incubated at 37° C. for 1 hour. The number of colony forming units determined to be present in each transformation reaction (i.e., per 100 ng pUC19 DNA) is indicated in Table 1.

TABLE 1

| | BL3496 | DH5α ™(Invitrogen) |
|---|---|---|
| +SAM | 471 | 0 |
| −SAM | 891 | 272 |

### Other Embodiments

The foregoing description is intended to illustrate and not limit the scope of the invention, which is defined by the scope of the appended claims. Other aspects, advantages, and modifications are within the scope of the following claims.

What is claimed is:

1. An isolated bacterium comprising: an F′ episome that confers high efficiency transformability; one or more mutations that allow transformation of methylated nucleic acids; one or mutations that allow transformation with unmethylated nucleic acids; and one or more mutations that confer resistance to bacteriophage infection.

2. The bacterium of claim 1 wherein said bacterium is an Escherichia coli strain.

3. The bacterium of claim 2 wherein said strain is K-12.

4. The bacterium of claim 3 wherein the genotype of said F′ episome comprises proAB⁺ lacI^{q} lacZΔM15 Tn10(Tet^{R}).

5. The bacterium of claim 3 wherein said one or more mutations that allow transformation of unmethylated nucleic acids involve the hsdR, hsdM and/or hsdS genes.

6. The bacterium of claim 3 wherein said one or more mutations that allow transformation of methylated nucleic acids involve the mcrA, mcrBC and/or mrr genes.

7. The bacterium of claim 3 wherein said bacteriophage is phage T1.

8. The bacterium of claim 3 wherein said one or more mutations that confer resistance to bacteriophage infection involve the tonA gene.

9. An isolated Escherichia coli K-12 bacterium, said bacterium having a genotype comprising mcrA Δ(mrr-hsdRMS-mcrBC) tonA/F′ proAB⁺ lacI^{q} lacZΔM15 Tn10(Tet^{R}).

10. The bacterium of claim 9, wherein said genotype comprises: mcrA Δ(mrr-hsdRMS-mcrBC) φ80(lacZ)ΔM15 Δ(lacZYA-argF) U169 endA1 recA1 supE44 thi-1 gyrA96 relA1 deoR tonA panD/F′ proAB⁺ lacI^{q} lacZΔM15 Tn10(Tet^{R}).

11. The bacterium of claim 9, wherein said bacterium is Escherichia coli BRL3946 (NRRL accession No. B-30640).

12. The bacterium of claim 9, wherein said bacterium is a derivative of Escherichia coli BRL3946 (NRRL accession No. B-30640).
